(19) United States
(12) Patent Application Publication
(10) Pub. No.: US 2013/0269416 A1
(43) Pub. Date: Oct. 17, 2013

(54) Method and device for combined measurement of bubbles and flow rate in a system for enriching a bodily fluid with a gas

(71) Applicant: THEROX, INC., (US)

(72) Inventors: Stephen E. Myrick, Tustin, CA (US); Jeffrey L. Creech, Los Angeles, CA (US); Terry A. Marchwick, Mission Viejo, CA (US)

(73) Assignee: THEROX, INC.

(21) Appl. No.: 13/815,602

(22) Filed: Mar. 12, 2013

Related U.S. Application Data
(63) Continuation-in-part of application No. 12/328,680, filed on Dec. 4, 2008.

(51) Int. Cl. G01N 29/02; A61M 1/36

(52) U.S. Cl. CPC G01N 29/02; A61M 1/3626
USPC 73/19.03

(57) ABSTRACT

This invention discloses a modular system having a base module, a mid-section control module, and a display module for preparing and administering a gas-enriched bodily fluid. Gas-enrichment is achieved by a gas-enriching device which can be in the form of a disposable cartridge. During operation, the gas-enrichment device is placed in an enclosure within the control module. An electronic controller manages the various aspects of the system such as the production of gas-enriched fluid, flow rates, bubble detection, and automatic operation and shut down. The system includes a combination bubble detector/flow meter that uses a single ultrasonic probe for measuring both bubbles and fluid flow rate.

### RELATED APPLICATIONS

This application is a continuation-in-part of U.S. patent application Ser. No. 12/328,680, filed on Dec. 4, 2008, entitled “System for Enriching a Bodily Fluid with a Gas Having a Removable Gas-Enrichment Device with an Information Recording Element,” by Myrick et al, which is incorporated herein by reference.

### FIELD OF THE INVENTION

The present invention relates generally to gas-enriched fluids and, more particularly, to a system that enriches a bodily fluid with a gas.

### BACKGROUND OF THE RELATED ART

This section is intended to introduce the reader to various aspects of art that may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Gas-enriched fluids are used in a wide variety of medical, commercial, and industrial applications. Depending upon the application, a particular type of fluid is enriched with a particular type of gas to produce a gas-enriched fluid having properties that are superior to the properties of either the gas or fluid alone for the given application. The techniques for delivering gas-enriched fluids also vary dramatically, again depending upon the particular type of application for which the gas-enriched fluid is to be used.

Many commercial and industrial applications exist. As one example, beverages may be purified with the addition of oxygen and carbonated with the addition of carbon dioxide. As another example, the purification of wastewater is enhanced by the addition of oxygen to facilitate aerobic biological degradation. As yet another example, in fire extinguishers, an inert gas, such as nitrogen, carbon dioxide, or argon, may be dissolved in water or another suitable fluid to produce a gas-enriched fluid that expands on impact to extinguish a fire.

While the commercial and industrial applications of gas-enriched fluids are relatively well known, gas-enriched fluids are continuing to make inroads in the healthcare industry. Oxygen therapies, for instance, are becoming more popular in many areas. A broad assortment of treatments involving oxygen, ozone, H₂O₂, and other active oxygen supplements has gained practitioners among virtually all medical specialties. Oxygen therapies have been utilized in the treatment of various diseases, including cancer, AIDS, and Alzheimer's. Ozone therapy, for instance, has been used to treat several million people in Europe for a variety of medical conditions including eczema, gangrene, cancer, stroke, hepatitis, herpes, and AIDS. Such ozone therapies have become popular in Europe because they tend to accelerate the oxygen metabolism and stimulate the release of oxygen in the bloodstream.

Oxygen is a crucial nutrient for human cells. It produces energy for healthy cell activity and acts directly against foreign toxins in the body. Indeed, cell damage may result from oxygen deprivation for even brief periods of time, and such cell damage can lead to organ dysfunction or failure. For example, heart attack and stroke victims experience blood flow obstructions or divergences that prevent oxygen in the blood from being delivered to the cells of vital tissues. Without oxygen, these tissues progressively deteriorate and, in severe cases, death may result from complete organ failure. However, even less severe cases can involve costly hospitalization, specialized treatments, and lengthy rehabilitation.

Blood oxygen levels may be described in terms of the concentration of oxygen that can be achieved in a saturated solution at a given partial pressure of oxygen (pO₂). Typically, for arterial blood, normal oxygen levels, i.e., normoxia or normoxemia, range from 90 to 110 mmHg. Hypoxemic blood, i.e., hypoxemia, is arterial blood with a pO₂ less than 90 mmHg. Hyperoxemic blood, i.e., hyperoxemia or hyperoxia, is arterial blood with a pO₂ greater than 400 mmHg, but less than 760 mmHg. Hyperbaric blood is arterial blood with a pO₂ greater than 760 mmHg. Venous blood, on the other hand, typically has a pO₂ level less than 90 mmHg. In the average adult, for example, normal venous blood oxygen levels range generally from 40 mmHg to 70 mmHg.

Blood oxygen levels also may be described in terms of hemoglobin saturation levels. For normal arterial blood, hemoglobin saturation is about 97% and varies only as pO₂ levels increase. For normal venous blood, hemoglobin saturation is about 75%. Indeed, hemoglobin is normally the primary oxygen carrying component in blood. However, oxygen transfer takes place from the hemoglobin, through the blood plasma, and into the body's tissues. Therefore, the plasma is capable of carrying a substantial quantity of oxygen, although it does not normally do so. Thus, techniques for increasing the oxygen levels in blood primarily enhance the oxygen levels of the plasma, not the hemoglobin.

The techniques for increasing the oxygen level in blood are not unknown. For example, naval and recreational divers are familiar with hyperbaric chamber treatments used to combat the bends, although hyperbaric medicine is relatively uncommon for most people. Since hemoglobin is relatively saturated with oxygen, hyperbaric chamber treatments attempt to oxygenate the plasma. Such hyperoxygenation is believed to invigorate the body's white blood cells, which are the cells that fight infection. Hyperbaric oxygen treatments may also be provided to patients suffering from radiation injuries. Radiation injuries usually occur in connection with treatments for cancer, where the radiation is used to kill the tumor. Unfortunately, at present, radiation treatments also injure surrounding healthy tissue as well. The body keeps itself healthy by maintaining a constant flow of oxygen between cells, but radiation treatments can interrupt this flow of oxygen. Accordingly, hyperoxygenation can stimulate the growth of new cells, thus allowing the body to heal itself.

Radiation treatments are not the only type of medical therapy that can deprive cells from oxygen. In patients who suffer from acute myocardial infarction, for example, if the myocardium is deprived of adequate levels of oxygenated blood for a prolonged period of time, irreversible damage to the heart can result. Where the infarction is manifested in a heart attack, the coronary arteries fail to provide adequate blood flow to the heart muscle. The treatment for acute myocardial infarction or myocardial ischemia often involves performing angioplasty or stenting of vessels to compress, ablate, or otherwise treat the occlusions within the vessel walls. In an angioplasty procedure, for example, a balloon is placed into the vessel and inflated for a short period of time to increase the size of the interior of the vessel. When the balloon is deflated, the interior of the vessel will, hopefully, retain most or all of this increase in size to allow increased blood flow.

However, even with the successful treatment of occluded vessels, a risk of tissue injury may still exist. During percutaneous transluminal coronary angioplasty (PTCA), the balloon inflation time is limited by the patient's tolerance to ischemia caused by the temporary blockage of blood flow through the vessel during balloon inflation. Ischemia is a condition in which the need for oxygen exceeds the supply of oxygen, and the condition may lead to cellular damage or necrosis. Reperfusion injury may also result, for example, due to slow coronary reflow or no reflow following angioplasty. Furthermore, for some patients, angioplasty procedures are not an attractive option for the treatment of vessel blockages. Such patients are typically at increased risk of ischemia for reasons such as poor left ventricular function, lesion type and location, or the amount of myocardium at risk. Treatment options for such patients typically include more invasive procedures, such as coronary bypass surgery.

To reduce the risk of tissue injury that may be associated with treatments of acute myocardial infarction and myocardial ischemia, it is usually desirable to deliver oxygenated blood or oxygen-enriched fluids to the tissues at risk. Tissue injury is minimized or prevented by the diffusion of the dissolved oxygen from the blood to the tissue. Thus, in some cases, the treatment of acute myocardial infarction and myocardial ischemia includes perfusion of oxygenated blood or oxygen-enriched fluids. The term “perfusion” is derived from the French verb “perfuse” meaning “to pour over or through.” In this context, however, perfusion refers to various techniques in which at least a portion of the patient's blood is diverted into an extracorporeal circulation circuit, i.e., a circuit which provides blood circulation outside of the patient's body. Typically, the extracorporeal circuit includes an artificial organ that replaces the function of an internal organ prior to delivering the blood back to the patient. Presently, there are many artificial organs that can be placed in an extracorporeal circuit to substitute for a patient's organs. The list of artificial organs includes artificial hearts (blood pumps), artificial lungs (oxygenators), artificial kidneys (hemodialysis), and artificial livers.

Increased blood oxygen levels also may cause hypercontractility in normally perfused left ventricular cardiac tissue to increase blood flow further through the treated coronary vessels. The infusion of oxygenated blood or oxygen-enriched fluids may follow the completion of PTCA or other procedures, such as surgery, to accelerate the reversal of ischemia and to facilitate recovery of myocardial function.

Conventional methods for the delivery of oxygenated blood or oxygen-enriched fluids to tissues involve the use of blood oxygenators. Such procedures generally involve withdrawing blood from a patient, circulating the blood through an oxygenator to increase blood oxygen concentration, and then delivering the blood back to the patient. There are drawbacks, however, to the use of conventional oxygenators in an extracorporeal circuit. Such systems typically are costly, complex, and difficult to operate. Often, a qualified perfusionist is required to prepare and monitor the system. A perfusionist is a skilled health professional specifically trained and educated to operate as a member of a surgical team responsible for the selection, setup, and operation of an extracorporeal circulation circuit. The perfusionist is responsible for operating the machine during surgery, monitoring the altered circulatory process closely, taking appropriate corrective action when abnormal situations arise, and keeping both the surgeon and anesthesiologist fully informed. In addition to the operation of the extracorporeal circuit during surgery, perfusionists often function in supportive roles for other medical specialties to assist in the conservation of blood and blood products during surgery and to provide long-term support for patient's circulation outside of the operating room environment. Because there are currently no techniques available to operate and monitor an extracorporeal circuit automatically, the presence of a qualified perfusionist, and the cost associated therewith, is typically required.

Conventional extracorporeal circuits also exhibit other drawbacks. For example, extracorporeal circuits typically have a relatively large priming volume. The priming volume is typically the volume of blood contained within the extracorporeal circuit, i.e., the total volume of blood that is outside of the patient's body at any given time. For example, it is not uncommon for the extracorporeal circuit to hold one to two liters of blood for a typical adult patient. Such large priming volumes are undesirable for many reasons. For example, in some cases a blood transfusion may be necessary to compensate for the blood temporarily lost to the extracorporeal circuit because of its large priming volume. Also, heaters often must be used to maintain the temperature of the blood at an acceptable level as it travels through the extracorporeal circuit. Further, conventional extracorporeal circuits are relatively difficult to turn on and off. For instance, if the extracorporeal circuit is turned off, large stagnant pools of blood in the circuit might coagulate.

In conventional extracorporeal circuits such as those used for heart-lung machines, the tubing and oxygenation device forming the circuit must be primed with a relatively large volume of a biocompatible fluid. As fluid flows through the circuit, which are generally lengthy and may have a tortuous fluid flow path, degassing, bubble formation or bubble trapping invariably results. Thus, additional measures must be taken to eliminate air bubbles from the circuit. For example, vacuum-based gas eliminator, or membrane-based bubble filters are typically required in conventional extracorporeal circuits and devices. In this regard, it is desirable to develop a fluid circuit that do not require large volume of fluid. Particularly, it is desirable to develop a fluid circuit with a fluid path that has a geometry that is smooth and conducive to streamlined laminar flow, thereby reducing the risk of bubble formation due to turbulent flow conditions.

In addition to the drawbacks mentioned above, in extracorporeal circuits that include conventional blood oxygenators, there is a relatively high risk of inflammatory cell reaction and blood coagulation due to the relatively slow blood flow rates and large blood contact surface area of the oxygenators. For example, a blood contact surface area of about one to two square meters and flow velocities of less than 5 centimeters/second are not uncommon with conventional oxygenator systems. Thus, relatively aggressive anticoagulation therapy, such as heparinization, is usually required as an adjunct to using the oxygenator.

Finally, perhaps one of the greatest disadvantages to using conventional blood oxygenation systems relates to the maximum partial pressure of oxygen (pO₂) that can be imparted to the blood. Conventional blood oxygenation systems can prepare oxygen-enriched enriched blood having a partial pressure of oxygen of about 500 mmHg. Thus, blood having pO₂ levels near or above 760 mmHg, i.e., hyperbaric blood, cannot be achieved with conventional oxygenators.

It is desirable to deliver gas-enriched fluid to a patient in a manner which prevents or minimizes bubble nucleation and formation upon infusion into the patient. The maximum concentration of dissolved gas achievable in a liquid is ordinarily governed by Henry's Law. At ambient temperature, the relatively low solubility of many gases, such as oxygen or nitrogen, within a liquid, such as water, produces a low concentration of the gas in the liquid. However, such low concentrations are typically not suitable for treating patients as discussed above. Rather, it is advantageous to use a gas concentration within a liquid that greatly exceeds its solubility at ambient pressure. Compression of a gas and liquid mixture at a high pressure can be used to achieve a high dissolved gas concentration according to Henry's Law, but disturbance of a gas-saturated or a gas-supersaturated liquid by attempts to inject it into an environment at ambient pressure from a high pressure reservoir ordinarily results in cavitation inception at or near the exit port. The rapid evolution of bubbles produced at the exit port vents much of the gas from the liquid, so that a high degree of gas-supersaturation no longer exists in the liquid at ambient pressure outside the high-pressure vessel. In addition, the presence of bubbles in the effluent generates turbulence and impedes the flow of the effluent beyond the exit port. Furthermore, the coalescence of gas bubbles in blood vessels may tend to occlude the vessels and result in a gaseous embolism that causes a decrease in local circulation, arterial hypoxia, and systemic hypoxia.

In gas-enriched fluid therapies, such as oxygen therapies involving the use of hyperoxic or hyperbaric blood, delivery techniques are utilized to prevent or minimize the formation of cavitation nuclei so that clinically significant bubbles do not form within a patient's blood vessels. However, it should be understood that any bubbles that are produced tend to be very small in size, so that a trained operator would typically have difficulty detecting bubble formation without the assistance of a bubble detection device. Unfortunately, known bubble detectors are ineffective for detecting bubbles in an extracorporeal circuit for the preparation and delivery of hyperoxemic or hyperbaric blood.

This problem results from the fact that the hyperbaric solutions can produce bubbles of the size beyond the resolution of known bubble detectors. Therefore, micro bubbles (bubbles with diameters of about 1 micrometer to about 1000 micrometers) may escape detection. Further, it is necessary not only to detect but to measure the size of these microbubbles, so that a cumulative bubble volume can be measured and the system can stop hyperoxemic blood delivery when a cumulative bubble volume threshold has been reached.

The present invention provides a system that can address the draw backs discussed above.

### SUMMARY OF THE INVENTION

In one aspect, the present invention provides a gas-enrichment system for enriching a bodily fluid with a gas-enriched fluid in an extracorporeal circuit. The system includes a combination bubble detector/flow meter having an ultrasonic probe that can generate a single ultrasonic signal for measuring both the bubbles as well as the flow rate of the returning bodily fluid in the system. The ultrasonic probe generates an ultrasonic signal through the combination bubble detector/flow meter. The system also includes a signal processing unit for processing the signal received from the ultrasonic probe. The signal processing unit uses the ultrasonic signal to measure bubbles and the flow rate of the returning bodily fluid. The signal processing unit is capable of deconvoluting the received signal into a flow rate (bulk velocity) measurement component and a bubble measurement component.

In another aspect, the present invention provides a method for measuring bubbles and fluid rate in a fluid along a fluid path utilizing a single ultrasonic signal in an extracorporeal circuit of the gas enrichment system. The method includes the general steps of: providing an ultrasonic probe for generating a probing ultrasonic signal directed at the fluid; transmitting a probing signal to the fluid and receiving a return signal from the fluid;

de-convoluting the return signal into a bubble signal component and a flow rate signal component; and measuring a flow rate and bubbles based on the de-convoluted return signal.

Other aspects and advantages of the invention will be apparent from the following description and the appended claims.

### BRIEF DESCRIPTION OF THE DRAWINGS

FIG. 1 shows a block diagram for an exemplary gas-enrichment system in accordance with embodiments of the present invention.

FIG. 2 shows the perspective view of an exemplary modular gas-enrichment system in accordance with embodiments of the present invention.

FIG. 3 shows a front view of the complete mid-section control module in the system of FIG. 2.

FIG. 4 shows a detailed view of an exemplary gas-enrichment device.

FIG. 5 shows a schematic view of an exemplary extracorporeal circuit setup in accordance with embodiments of the present invention.

FIG. 6 shows a state diagram of an exemplary system software in accordance with embodiments of the present invention.

FIG. 7 shows a detailed view of the mixing chamber in the gas-enrichment device in accordance with embodiments of the present invention.

FIG. 8 shows a block diagram illustrating the components of the electronic circuit operating the bubble detector/flow meter in accordance with embodiments of the present invention.

FIG. 9A, 9B, and 9C show a representation of the signal transmitted by the ultrasonic probe, received by the ultrasonic probe, and the comparison of upstream/downstream signals, respectively.

FIG. 10 shows a block diagram illustrating the algorithm for bubble detection and measurement in accordance with embodiments of the present invention.

FIG. 11 shows a block diagram illustrating the algorithm for flow rate measurement in accordance with embodiments of the present invention.

### DETAILED DESCRIPTION

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

System Overview

While systems of the present invention are not limited to preparation of any particular type of gas or bodily fluid, for the purpose of illustration, the following discussion will use the preparation of oxygen-supersaturated fluids and the administration of Supersaturated Oxygen (SSO₂) Therapy as an example.

For the purpose of the present discussion, SSO₂ Therapy refers to minimally invasive procedures for enriching oxygen content of blood through catheter-facilitated infusion of oxygen-supersaturated physiological fluid. These procedures generally are aimed at treating the culprit vessel of an acute myocardial infarction (AMI) after successful percutaneous intervention (PCI) with stenting has been performed.

In a preferred embodiment, a system for administering SSO₂ Therapy generally includes three component devices: the main control system, the oxygen-enrichment device (i.e., the oxygenator), and the infusion device (e.g., an SSO₂ delivery catheter). These devices function together to create a highly oxygen-enriched saline solution called SuperSaturated Oxygen (“SSO₂”) solution. A small amount of autologous blood is mixed with the SSO₂ solution producing oxygen-supersaturated blood, and then delivered to the targeted major epicardial artery via the SSO₂ delivery catheter. Typical duration of SSO₂ Therapy is 60 to 90 minutes.

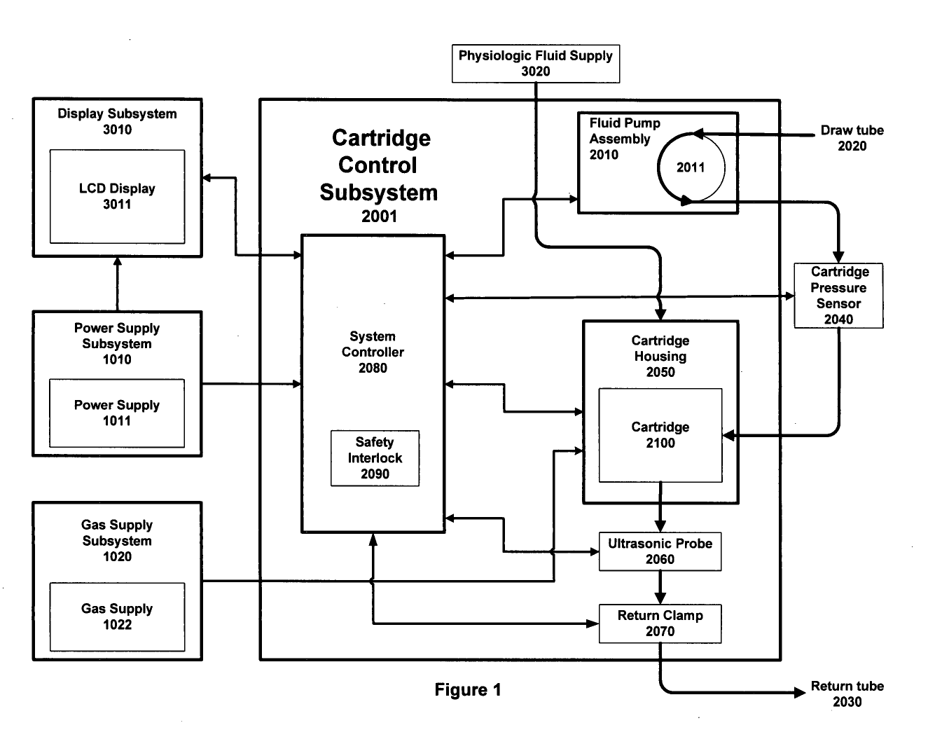

Starting with the overall system that contains the combination bubble detector/flow meter, FIG. 1 shows a block diagram that illustrates the architecture of a system in accordance with embodiments of the present invention. With reference to FIG. 1, systems in accordance with embodiments of the present invention are generally organized into a number of key subsystems, including a Display subsystem 3010 which typically comprises a display 3011, such as a LCD display for providing a user interface to the system, a Power Supply subsystem 1010 which typically comprises a power supply 1011 for providing power to the system, a Gas Supply subsystem 1020 which typically comprises a gas supply 1022 (such as an oxygen bottle) for supplying the system with a gas to be used in enriching the fluid, and a Cartridge Control subsystem 2001 for automatic control of gas-enrichment and other system operations. Although a detailed discussion of the gas-enrichment device will be provided later, it is important to recognize that while in this preferred embodiment the gas-enrichment device is in the form of cartridge 2100 (hence the name Cartridge Control subsystem), it is not the only possible form for the gas-enrichment device.

Functionally, the Cartridge Control subsystem 2001 is the centerpiece of the entire system. As shown in FIG. 1, the Cartridge Control subsystem typically comprises a system controller 2080 for processing input information and issuing commands to the various components of the system. In a preferred embodiment, the system controller 2080 also incorporates safety interlock 2090 circuitry for monitoring and ensuring the system operates within safety parameters. The safety interlock 2090 may be implemented with a logic device such as field programmable gate arrays (FPGA).

The Cartridge Control subsystem 2001 also includes a fluid pump assembly 2010 which typically comprises a fluid pump 2011, a draw tube 2020, a cartridge pressure sensor 2040, the gas-enrichment device (e.g. cartridge) 2100, an ultrasonic bubble detector/flow meter (i.e. ultrasonic probe) 2060, a return clamp 2070, a return tube 2030. The cartridge housing 2050 is configured to receive the cartridge 2100 (i.e. the gas-enrichment device). Within the cartridge housing 2050 enclosure, various sensing, controlling, and interfacing mechanisms are provided for use with the cartridge 2100. Physiologic fluid supply 3020, such as an IV bag, is included to provide a physiologic fluid source to the system.

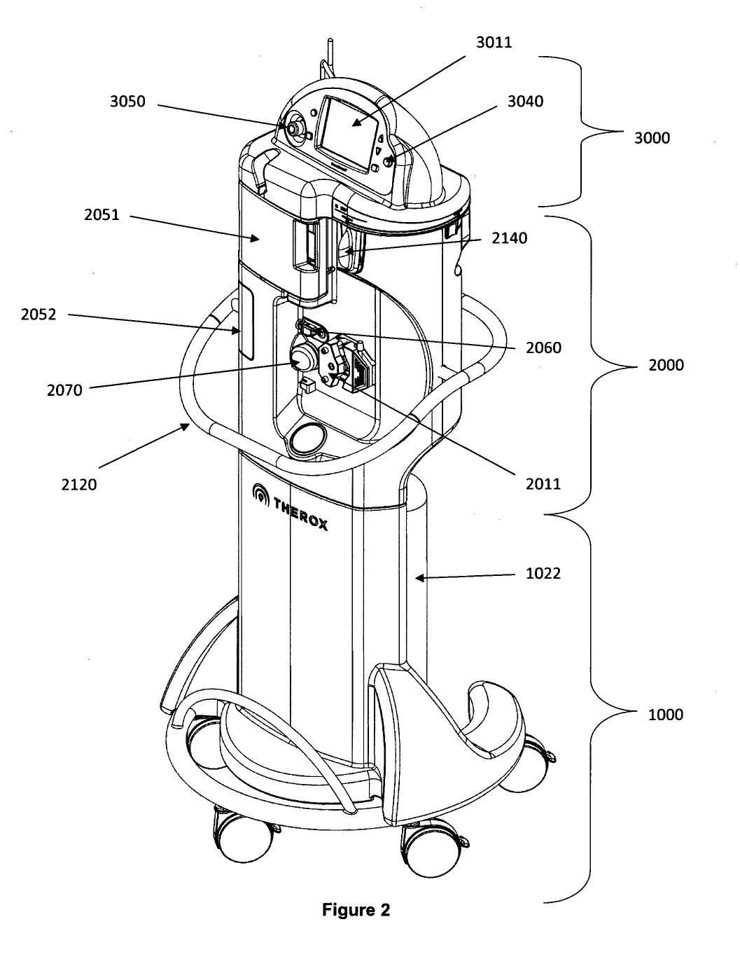

It will be understood by those skilled in the art that this organizational architecture is a conceptual architecture. Actual physical implementation is a matter of design choice which may take on various physical forms. For example, FIG. 2 shows a preferred implementation of a system in accordance with the system architecture of FIG. 1. As shown in FIG. 2, the system has a modular design comprising three modules, the base module 1000, the mid-section control module 2000, and the display module 3000. Referring back to FIG. 1, the Display subsystem 3010 provides an access point to the host/user interface of the system.

The base module 1000 shown in FIG. 2 may provide system mobility with wheels, and may also house batteries for mobile operation and power supplies for conversion of facility electric power for use by the system. The Gas Supply subsystem 1020 shown in FIG. 1, also may be housed in the base module 1000 shown in FIG. 2. Although some of the electrical and/or mechanical components of the system may be housed in the base module 1000, these components may be placed in alternate locations of the system as a matter of design choice. To facilitate positioning of the system, a handle 2120 may be coupled to the mid-section control module 2000 for directing movement of the system.

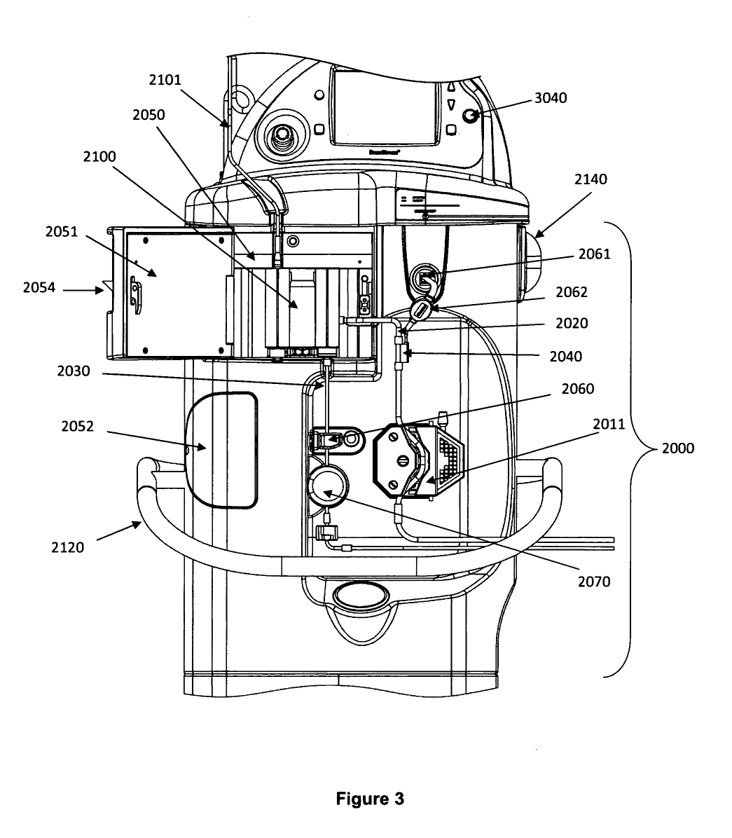

Each of the three modules may include doors or access panels for protecting and accessing the various components housed therein. For example, FIG. 3 shows the mid-section control module 2000 as having an enclosure with a hinged housing door 2051 for enclosing the cartridge (i.e. gas-enrichment device) 2100 and access panel 2052 for covering the access window to the internal space of the module. This cartridge housing 2050, shown in FIG. 3, is preferably an anodized aluminum enclosure. As shown in FIG. 3, the cartridge housing assembly is embedded in the mid-section control module 2000.

Continuing to refer to FIG. 3, a modular jack 2061 located on the front of the system enclosure is provided for connecting the cartridge pressure sensor 2040 to the system controller 2080 (not shown) via a cartridge pressure sensor cable 2062. During setup, the system user inserts the cartridge pressure sensor cable 2062 into the modular jack 2061 on the front of the system main enclosure.

FIG. 3 shows the configuration in which the cartridge 2100 is loaded in the cartridge housing 2050. The cartridge housing 2050 has a door 2051 and grooves for fitting the draw tube 2020 and return tube 2030. The cartridge housing 2050 works in conjunction with the cartridge 2100 during operation. It has a motorized piston actuator that operates the cartridge piston and delivers saline from an IV bag. The cartridge housing 2050 also has a set of needle valve actuators to control the flow of liquid through the cartridge 2100, and vent valve actuators to depressurize the cartridge oxygen chamber and mixing chamber.

Continuing to refer to FIG. 3, pulling the cartridge housing door handle 2054 down and forward opens the housing door 2051 when it is unlocked. An indicator is illuminated when the housing door 2051 is unlocked and ready to be opened. After the housing door 2051 is opened, the user may insert the cartridge 2100 into the cartridge housing 2050 enclosure. Slots in the cartridge housing 2050 enable passage of the draw tube 2020, return tube 2030 and IV tube 2101. When the housing door 2051 is closed, the cartridge 2100 is automatically aligned with all mechanical and sensor interfaces within the cartridge housing 2050 when the housing door 2051 is closed. In operation, the pressurized chambers of the cartridge 2100 are enclosed within the cartridge housing 2050.

Referring again to FIG. 1, a solenoid-operated oxygen valve controls the flow of oxygen from the Gas Supply subsystem 1020 to the cartridge 2100. The valve is normally closed. The valve is pulsed open in feedback with the oxygen pressure transducer to maintain the oxygen pressure in the cartridge 2100 at the desired set point (of approximately 600 psi).

Referring again to FIG. 3, an appropriate draw tube 2020 is used to draw a bodily fluid (e.g. blood) from a patient. The drawing action is provided by the fluid pump assembly 2010. Specifically, the fluid pump assembly includes a fluid pump 2011, such as a peristaltic pump. As the peristaltic pump 2011 mechanically produces a driving force along the flexible draw tube 2020, fluid within the draw tube is pumped in the direction towards the cartridge 2100 in the system. After the fluid is oxygenated within the cartridge 2100, the fluid is returned to the patient through the return tube 2030. The ultrasonic probe 2060 is typically coupled to the return tube 2030. The construction of the return tube is not particularly limited, as long as the tubing can be clamped into the ultrasonic probe and can transmit ultrasonic signals. Medical grade materials such as polyvinyl chloride (PVC), for example may be used. Feedback from the ultrasonic probe 2060 can be used with a fluid pump assembly 2010 to operate as an automatic extracorporeal circuit that can adjust the speed of the fluid pump 2011 to maintain the desired flow rate as well as provide continuous monitoring of operating parameters to ensure safety conditions are met.

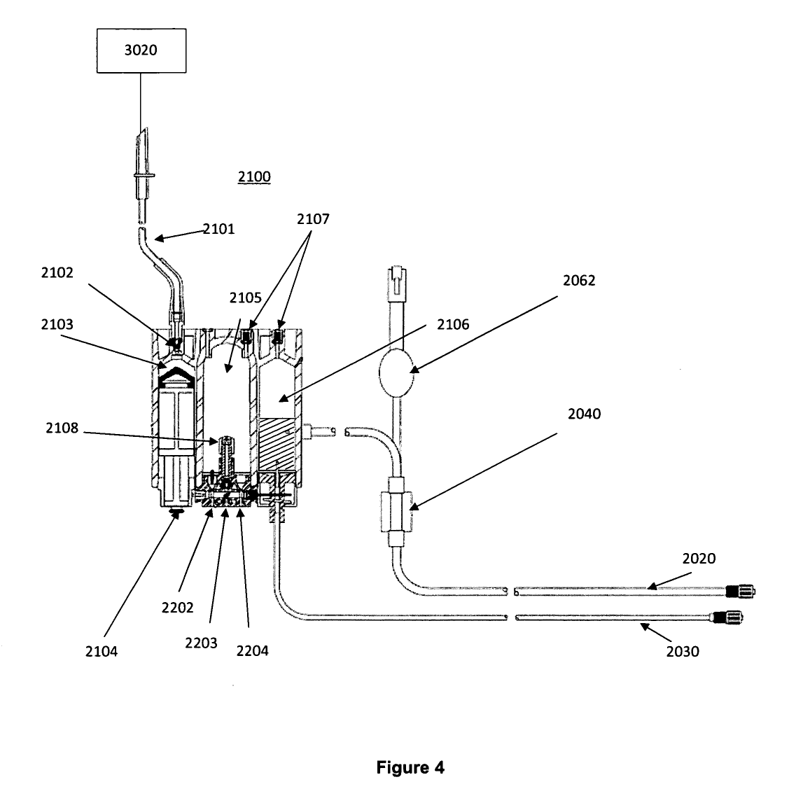

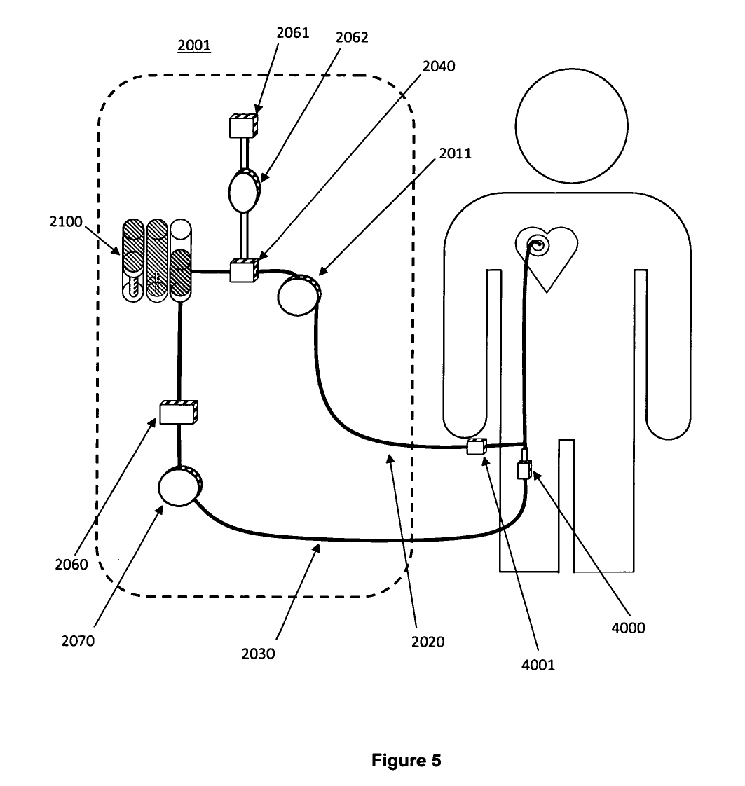

In one embodiment of the present invention, the gas-enrichment device is in the form of a cartridge 2100, as shown FIG. 4. The cartridge 2100 is a single-use disposable device that is designed to be loaded into the system. The cartridge 2100 has a three-chambered body that creates SSO₂ solution from inputs of hospital-supplied oxygen and physiologic saline, and mixes the SSO₂ solution with arterial blood within the cartridge blood path. As shown in FIG. 5, the cartridge 2100 has a tube set that draws the patient's arterial blood through the draw tube 2020, and returns oxygen-supersaturated blood through the return tube 2030 to the SSO₂ delivery catheter 4000. The cartridge draw tube 2020 connects to an arterial sheath 4001. Sheath placement may be coaxial (single arterial access site) or contralateral (two arterial access sites) at the physician's discretion. A physician makes two tubing connections during the initiation of SSO₂ therapy. The cartridge draw tube 2020 is attached to the arterial sheath 4001 before priming the blood flow path, and the return tube 2030 is attached to the SSO₂ delivery catheter 4000 after the blood flow path is successfully primed.

As shown in FIG. 5, an SSO₂ delivery catheter (the infusion device) 4000 is connected to the return tube 2030 for conducting the oxygen-enriched blood back to the patient. The SSO₂ delivery catheter 4000 is not particularly limited. Any suitable SSO₂ delivery catheter known in the art may be advantageously used. Materials such as polyethylene or PEBAX (polyetheramide), for example, may be used in the construction of the catheter. Also, the lumen of the catheter should be relatively free of transitions that may cause the creation of cavitation nuclei. For example, a smooth lumen having no fused polymer transitions typically works well.

Still referring to FIG. 5, the draw tube 2020 connects to the same femoral arterial sheath 4001 that may be used for angioplasty and stenting procedures. Sheath placement may be coaxial (in one femoral artery) or contralateral (in both the right and left femoral arteries), at the physician's discretion. FIG. 5 illustrates how arterial blood is withdrawn via the sidearm of the sheath through the annular space between the SSO₂ delivery catheter 4000 and sheath 4001; in this configuration (coaxial), a single introducer sheath can be used. The draw tube 2020 luer fitting connects to the sidearm of the sheath. The SSO₂ delivery catheter is placed through the axial port of the sheath, to the desired target location. When extracorporeal blood flow is initiated, the catheter and the return tube 2030 are wet-connected to ensure that no gaseous emboli are introduced to the patient during priming. The term ‘wet connection’ requires that both devices are fully blood-primed and free of trapped air bubbles. The return tube 2030 luer fitting connects to the luer hub of the SSO₂ delivery catheter 4000. For the contralateral approach (not shown), a smaller introducer sheath is used for the draw side access, while a second introducer sheath provides access for the catheter. This alternative approach may be used by physicians who prefer to use two smaller sheaths for arterial access instead of a single sheath.

It should be noted that one advantageous feature of the present invention is that the fluid path may be configured to minimize bubble formation without requiring additional bubble-removing apparatus. Exemplary geometric parameters that may affect the internal fluid flow path bubble nucleation characteristics include the cross-sectional area of the fluid stream, slope or gradient of path curvature, and surface roughness, but are not limited thereto. These parameters may be easily incorporated in the modular design of an exemplary system of the present invention to achieve an anti-bubble forming fluid flow path geometry. In general, straight-line geometries, uniform flow path diameter, and slow changing curvatures are conducive to non-turbulent flows.

Turning our attention now to the cartridge (i.e. gas-enrichment device) 2100, FIG. 4 shows that the blood (bodily fluid) is to be pumped through the draw tube 2020 into the cartridge 2100. Although various different types of oxygenation devices may be suitable for oxygenating the patient's blood prior to its return, the cartridge 2100 of the present invention advantageously prepares an oxygen-supersaturated physiologic fluid and combines it with the blood to enrich the blood with oxygen. Also, the cartridge 2100 is advantageously sterile, removable, and disposable, so that after the procedure has been completed, the cartridge may be removed and replaced with another cartridge for the next patient.

The cartridge 2100 may additionally incorporate an information recording element to record the patient's health information and procedural data so that the cartridge is individually customized to guard against operator error. Exemplary information recording elements may include a barcode label, an RFID chip, a PROM, or any combinations thereof Other component features and advantages of the cartridge 2100 will be described in great detail below.

For the purposes of understanding FIGS. 1 and 4, it is sufficient at this point to understand that the physiologic fluid, such as saline, is delivered from a suitable physiologic fluid supply 3020, such as an IV bag, to a first chamber (i.e. piston chamber) 2103 within the cartridge 2100 under the control of a system controller 2080. A suitable gas, such as oxygen, is delivered from a gas supply 1022, such as an oxygen tank, to a second chamber (i.e. oxygen chamber) 2105 within the cartridge 2100. Generally speaking, the physiologic fluid from the first chamber 2103 is pumped into the second chamber 2105 and atomized to create an oxygen-supersaturated physiologic solution. This oxygen-supersaturated physiologic solution is then delivered into a third chamber (i.e. mixing chamber) 2106 of the cartridge 2100 along with the blood from the patient. As the patient's blood mixes with the oxygen-supersaturated physiologic solution, oxygen-enriched blood is created. This oxygen-enriched blood flows from the third chamber (i.e. mixing chamber) 2106 of the cartridge 2100 into the return tube 2030.

The patient connections of the draw tube 2020 and the return tube 2030 that couple to the cartridge (i.e. gas-enrichment device) 2100 are shown in FIG. 5. Referring back to FIG. 1, in this exemplary implementation, a cartridge pressure sensor 2040, which is operatively coupled to the Cartridge Control subsystem 2001, provides pressure readings to the system controller 2080. The location of one or more pressure sensors is not particularly limited so long as the pressure being measured corresponds to a desired measurement location within the extracorporeal circuit. In the current embodiment, the pressure sensor is located to measure peak pressure within the extracorporeal circuit, which is in the cartridge draw tube 2020 after the fluid pump 2011. In some embodiments, it is envisioned that load cells may be incorporated directly in the Cartridge Control subsystem 2001 so as to eliminate the need for disposable pressure sensors such as shown in the current embodiment. This configuration will have the benefit of reducing the cost of the disposable cartridge 2100.

The system controller 2080 may also receive signals from one or more level sensors that monitor the level of fluid within the mixing chamber 2106 (not shown) of the oxygenation device to ensure that proper fluid level is maintained for the oxygen-supersaturated physiological solution to mix with the patient's blood with little or no bubble formation.

The ultrasonic probe 2060, a combination bubble-detector/flow meter that clamps on the return tube 2030, represents another advantageous feature of the present system. Several conventional techniques are known in the art for measuring a flow rate of a liquid flowing in a conduit, pipe, or tube. These include thermal flow meters, coriolis force flow meters, differential pressure flow meters, and ultrasonic flow meters. Generally, fluid flow meters sense one or more parameters (e.g. volumetric or mass) of the flow that can be calibrated to correspond to the rate of fluid flow. In the case of volumetric flow, the measured parameter is often bulk velocity, which can be used to calculate flow rate based on the known cross section of the fluid path. Measuring flow rate is difficult for fluids having relatively slow flow velocities (e.g. <5 cm/s). Thus, the return tube size (and therefore ultrasonic probe size) is chosen to maximize the flow velocity within the tube without producing turbulence or excessive pressure drop in the return tube. For example, a circuit designed to operate at 100 ml/min would advantageously use a 3/32″ (0.24 cm) internal diameter tube to measure flow rates from 0-150 ml/min at velocities from 0-100 cm/sec (while maintaining laminar flow through the ultrasonic probe).

In situations where bubbles are present in the fluid, the presence of a bubble may disrupt any type of flow meter in addition to presenting a potential hazard. Thus, in conventional applications, fluid flow measurements are often coupled with gross bubble detection (air-in-line detection) to alert the user when bubbles are present or flow readings are not valid. However, conventional flow meters typically only detect but do not measure bubbles, because they are concerned primarily with the presence of the bubbles that will cause the loss of signal which in turn will interrupt the flow measurement, not in determining the bubble volume. In particular, microbubbles (bubbles from 1-1000 microns) do not disrupt flow measurement significantly and thus are typically not detected by conventional flow meters. Moreover, conventional flow meters do not attempt to measure bubble volumes, particularly microbubble volumes. Measuring bubble volume requires additional steps to determine individual bubble size, calculate bubble volume, and calculate the cumulative bubble volume. Larger bubbles do not need to be measured because their presence alone is enough to require a response by the system. The need to measure the cumulative bubble volume arises because an extracorporeal circuit, such as the present invention, can deliver highly oxygenated blood to a patient, and if not properly monitored can also deliver significant amounts of microbubbles to a patient over the course of treatment.

The present invention uses a single ultrasonic probe to both detect and measure bubbles, including microbubbles, and measure flow rate at the same time. As shown in FIG. 3, the ultrasonic probe 2060 is typically positioned at the return tube 2030 to detect bubbles as they pass through the return tube to the patient. The orientation of the ultrasonic probe with respect to the return tube 2030 will be discussed in greater detail below.

The system receives signals from the ultrasonic probe 2060 and processes information regarding the fluid flow rate and the nature of any bubbles that may be traveling in the oxygen-enriched blood going back to the patient. Data produced using the ultrasonic probe 2060 may also be used by the system to control or shut down the system in certain circumstances as discussed in detail below.

Another advantageous feature of the present inventive system is the automated priming mechanism and the small volume of priming fluid required. Relative to conventional extracorporeal circuits, the system has a far smaller priming volume requirement, typically in the range of 25 to 100 milliliters. Thus, a heater typically is not used with the system. In this preferred embodiment, priming may be initiated by holding down the priming switch 3040 shown in FIG. 2. When the priming switch 3040 is released, priming action is immediately stopped.

It will be noted that one advantageous feature of the present invention is the automated safety responses enabled by the system controller 2080 and safety interlock 2090 shown in FIG. 1. In conventional extracorporeal systems, occlusion in the fluid conduits such as tubes or catheters often occurs. Vigilant monitoring by the human operator is often required to catch such occlusion events and resolve the problem by manually stopping the system, removing the occlusion, and then restarting the system regardless of the degree of the occlusion. This indiscriminate occlusion resolution procedure is both time consuming and labor intensive. Systems of the present invention can monitor for occlusion events by measuring changes in flow rate, bubble activity, and/or extracorporeal circuit pressure, and respond according to the level of occlusion.

The details of the system and its various respective subsystems will now be described with reference to the preferred embodiment as illustrated in the figures.

The Cartridge Control Subsystem

Components of the Cartridge Control subsystem 2001 are mainly located in the mid-section of the system. As shown in FIGS. 1 and 3, components of this subsystem may include the system controller 2080, the cartridge housing 2050, the cartridge 2100 (when loaded), the fluid pump assembly 2010, the ultrasonic probe 2060, the cartridge pressure sensor 2040, and the return clamp 2070.

The system controller 2080 is the electronic assembly that monitors and controls the operation of the cartridge 2100 during SSO₂ Therapy. The electronic circuitry of the system controller 2080 is preferably implemented in a printed circuit board (PCB). The system controller PCB receives digital and analog signals from sensors and other electronic components within the system. The system controller 2080 has a microprocessor that controls the fluid pump assembly, the piston actuator and all solenoids of the Cartridge Control subsystem 2001. The system controller preferably uses an 8051 microprocessor with software encoded in persistent memory so that it will be available to the system upon power up.

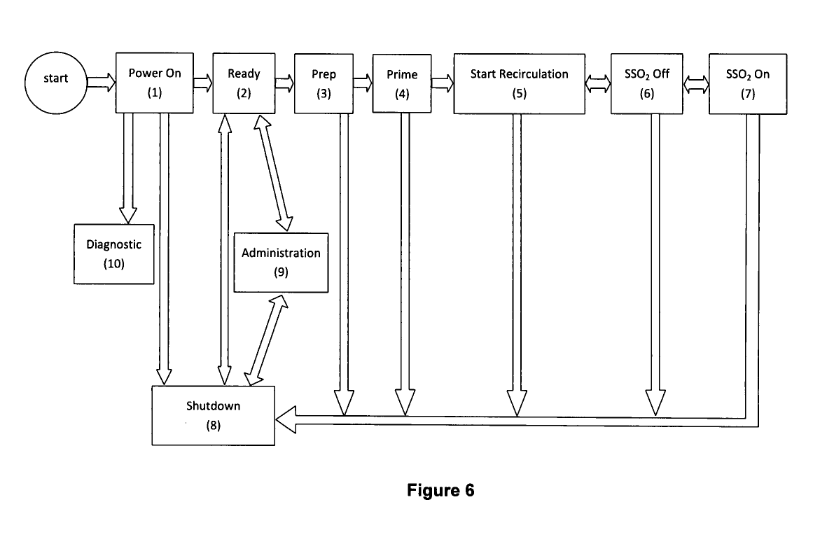

FIG. 6 shows a software state diagram of the control software. The software is automatically executed when the Cartridge Control subsystem is started. The control software provides ten different operating states, including: (1) Power On State; (2) Ready State; (3) Prep State; (4) Prime State; (5) Start Recirculation State; (6) SSO₂ Off State; (7) SSO₂ On State; (8) Shut Down State; (9) Administration State; and (10) Diagnostics Mode State. The lines and arrows connecting the various different states indicate the logical relationship and transition between the connected states. This software system is preferably implemented in C++.

With reference to FIG. 6, when the system is first powered up, the software first enters the Power On state (1), during which the system runs a series of initialization tests such as testing the power supply, the internal watchdog timer, etc. If a critical fault condition is detected during power up, the system enters the Shut Down state (8) to shutdown the system. The Power On state also offers the option to enter the Diagnostics Mode (10) which allows the user to perform service on the system with relaxed interlocks. Once power up is successfully executed, the system may enter into the Ready state (2).

The Ready state (2) is the state that performs Cartridge loading/unloading. If system encounters critical error during loading or unloading of the Cartridge, the system enters the Shut Down state (8). User input is then required to clear the loading/unloading error and take the system back to the Ready state (2). From either the Shut Down (8) or the Ready (2) state, the user can interrupt the system to enter the Administration state (9) in order to check system parameters and perform system administrative tasks.

After the cartridge has been loaded into the cartridge housing, the user has spiked the saline IV bag, and the cartridge pressure sensor is connected to the system, the user can Prep the cartridge. The purpose of Prep state (3) is to check the Fill, Flush and Flow Valves, to saline prime the fluid path in the high-pressure side of the cartridge, establish the minimum liquid level, and pressurize with oxygen. If system encounters critical error during Prep, the system enters the Shut Down state (8). Prep is fully automated after user initiation.

Upon user input, the system may enter the Prime state (4) to prime the extracorporeal circuit. During this state, the fluid pump is activated so long as the priming switch is pressed. Patient blood is drawn into the mixing chamber as the pump turns.

Again, the Prime state (4) also offers the option of taking the system into the Shut Down state (8) when a critical fault condition is encountered or when a user input to shutdown is received. All states following the prime state will have the same option of entering the Shut Down state (8) upon encountering a critical fault condition or user input. Once circuit priming is completed, the system software automatically enables flow measurement and bubble detection functionality.

After the user makes wet-to-wet connection of the return tube to the infusion device, the system enters the Recirculation state (5), during which the pump continues to turn as the blood flow and return pressure stabilize. Complete circulation of fluid in the extracorporeal circuit is established during this state to finish the priming process.

When priming is completed, the system enters into the SSO₂ OFF state (6), during which circulation of the extracorporeal circuit is continued with no infusion of SSO₂ solution. Thus, the blood from the patient is only diluted slightly with the un-oxygenated saline. This state also allows the option of detecting a non-critical fault condition such as a minor occlusion in the extracorporeal tubing. When such a non-critical fault condition is encountered, the system may return to the Recirculation state (5) to re-establish proper extracorporeal circulation. If no fault condition occurs, the system will remain in the SSO₂ OFF state until user input is received to enter the SSO₂ ON state (7).

In the SSO₂ ON state (7), the system produces oxygenated saline, infuses the oxygenated saline with patient's blood in the mixing chamber of the Cartridge, and then return the oxygenated blood back to the patient. The system will remain in this state until completion of the therapy, user interruption, or a fault condition. Similar to the SSO₂ OFF state (6), the SSO₂ ON state (7) also allows the option of responding to a minor non-critical fault condition by returning the system back to the previous state, thus avoiding unnecessary shutdown of the system.

As discussed above, and referring back to FIG. 1, the system controller 2080 incorporates a safety interlock 2090 that communicates with many of the components of the system for various reasons. The safety interlock 2090 monitors various components to ensure that the system is operating within certain prescribed bounds. For example, the safety interlock 2090 may receive information regarding extracorporeal circuit pressures from the pressure sensors, information regarding fluid level in the mixing chamber from the level sensor, as well as other information regarding the operating states of the various components. Based on this information, the safety interlock can shut down the system should it begin to operate outside of the prescribed bounds. For example, the safety interlock 2090 can stop the fluid pump 2011 and engage the return clamp 2070 on the return tube 2030 to stop flow of the fluid, as well as disable the fluid pump assembly 2010 and the system controller 2080 that controls the cartridge 2100.

While the safety interlock 2090 typically operates in this automatic fashion, a safety switch (e.g. an emergency stop switch 3050 shown in FIG. 2) may be provided so that a user can initiate a shutdown of the system in the same fashion even if the system is operating within its prescribed bounds.

Also included in the system controller 2080 circuitry is a safety interlock 2090 for monitoring and ensuring that system operates within safety parameters. Any number of hardware implementations may be used to implement the safety interlock. In this preferred embodiment, the safety interlock 2090 is implemented using a Field Programmable Gate Array (FPGA), which is integrated on the system controller PCB. The safety interlock 2090 continuously monitors inputs for events that require treatment stoppage or enable treatment. The safety interlock 2090 has the ability to disable all powered electronics in the system. When certain conditions occur, the safety interlock 2090 stops treatment by disabling powered (24V) electronics, which automatically closes the return clamp 2070, stops the fluid pump 2011, stops the piston 2104, closes the fluid flow valve 2204, closes the oxygen valve, and depressurizes the cartridge 2100. For instance, the analog cartridge pressure sensor 2040 is monitored by the FPGA; circuit pressures exceeding the threshold of 2000 mmHg (38 psig) will stop treatment.

Exemplary function-disabling parameters monitored by the safety interlock 2090 may include signals such as: emergency stop engaged, oxygen pressure high signal, piston pressure high signal, temperature high or low signal, low system voltage signal, fluid pump failure, piston failure, solenoid driver fault condition, display failure or bubble detector failure, and other relevant parameters.

The safety interlock 2090 also has logic that enables functions within the Cartridge Control subsystem 2001 based on monitored inputs. For instance, the fluid pump 2011 will not operate if the pump head is open. Exemplary function-enabling parameters monitored by the safety interlock may include: prime switch pressed, pump head open, cartridge door open, oxygen valve open, fluid pump operating, cartridge detected, cartridge pressure sensor detected, and other relevant parameters. In addition to the above mentioned inputs to the safety interlock 2090, a number of operating inputs may also be monitored by the system controller to influence system behavior, including inputs from the priming switch 3040, and inputs from the emergency stop switch. As shown in FIG. 2, the priming switch 3040 is mounted to the display module 3000. The user must press and hold to start the pump motor and initiate blood flow. The emergency stop (E-stop) switch 3050 is also mounted to the display module 3000. The system controller disables all powered electronics (24 VDC) upon manual actuation of the E-stop switch by the system user. The E-Stop switch latches when pressed and must be manually disengaged.

The Display Subsystem and the User Interface

In the present preferred embodiment as shown in FIG. 2, the Display subsystem is embodied in a modular casing, shown as the display module 3000. The Display subsystem provides the main user interface of the system, which preferably includes the control buttons (including the emergency stop switch 3050 and the priming switch 3040) and the LCD display 3011 for communicating with the user. An IV pole is also attached to provide a structure for hanging IV solution bags.

Referring to FIG. 2, the display module 3000 is shown as already mounted on the top of the mid-section control module 2000. In this figure, the system's power lever 2140 is located at the dividing line of the upper section and the mid-section. The power lever controls both the ON/OFF states of the entire system and also the OPEN/CLOSE states of the oxygen tank 1022. During operation, a user slidably moves the power lever 2140 to the side of the system (the ON position) in order to turn on the system.

Implementation of the Display subsystem includes the display PCB, control buttons, and the liquid crystal display (LCD) 3011. The display PCB is mounted within the display enclosure. The display PCB contains a microprocessor for operating the display and electronic circuitry for the control buttons. The display PCB preferably uses a commercially available microprocessor that provides the platform for the application software running a Linux-based operating system. This software communicates serially with the system controller 2080.

The display enclosure also houses the LCD. The LCD screen displays the video output of the display PCB. A set of control buttons located around the display provide the input for user selection of application software.

The Power Supply Subsystem

As shown in FIG. 1, the Power Supply subsystem 1010 is an electronic assembly that provides DC power to the various subsystems. The power supply 1011 receives power from the AC mains or internal batteries. Component features of the Power Supply subsystem 1010 may include, but not limited to, detachable power cord, appliance inlet receptacle with fuses and selection of 110-V or 220-V operation, isolation transformer, AC to DC power supply with fuse, battery with fuse, battery charger, and DC to DC power supplies. The Power Supply subsystem 1010 also includes specific DC power supplies that provide fixed voltages to other subsystems.

The Gas-Supply Subsystem

As discussed above, the system of the present invention may be used to prepare a number of different gas-enriched fluids. In this preferred embodiment, it is a Gas Supply subsystem 1020 that provides oxygen to the cartridge 2100.

The Fluid Pump Assembly

Referring again to FIG. 1, the Cartridge Control subsystem 2001 includes a fluid pump assembly 2010, which brings together the fluid pump 2011, the ultrasonic probe 2060, the draw tube 2020, the return tube 2030, and the return clamp 2070 to modulate fluid flow through the system.

When the system is used in SSO₂ therapy, the fluid pump 2011 withdraws arterial blood from the patient and pumps it through the cartridge 2100 and the SSO₂ delivery catheter 4000 back to the patient (see FIG. 5). The fully occlusive peristaltic fluid pump 2011 interfaces with the cartridge tubing and thus does not have direct fluid contact. The system user inserts the draw tube 2020 into the fluid pump head 2011 and the return tube 2030 into the ultrasonic probe during system set-up.

The Gas-enrichment Device (Cartridge)

FIG. 4 shows a schematic illustration of the three-chambered structure of the cartridge 2100, which includes the piston chamber 2103, the oxygen chamber 2105, and the mixing chamber 2106. In operation, saline is drawn from the IV bag 3020 into the piston chamber 2103, and then pumped into the pressurized oxygen chamber 2105. Oxygen is supplied from an oxygen tank 1022 (not shown) and introduced into the oxygen chamber 2105, mixing with the atomized saline to form an oxygen-supersaturated physiologic fluid. This oxygen-rich saline is then introduced into the mixing chamber 2106 to be mixed with blood. The blood is drawn into the mixing chamber 2106 through the draw tube 2020. Once the blood is mixed with the oxygen-rich saline, the mixture is then returned to the patient through the return tube 2030.

Referring to FIG. 4, during operation, a tube 2101 is coupled to the IV bag 3020 to provide saline. The other end of the tube is coupled to a port on the cartridge 2100, forming a passageway that leads to the piston chamber 2103. A check valve 2102 is disposed so that fluid may enter the piston chamber 2103 when the piston is pulled downward, but fluid cannot return to the IV bag 3020 when the piston is pushed upward. Referring again to FIG. 4, a piston 2104 is located at the opposite end of the piston chamber 2103 from the check valve 2102. The piston chamber 2103 has a second fluid passageway that is coupled to a fluid passageway located in the base of the oxygen chamber 2105 by a tube. The passageway in the base of the oxygen chamber is an inlet to a valve assembly that controls the manner in which fluid from the piston chamber 2103 is delivered into the oxygen chamber 2105. The valve assembly (or manifold) includes three needle valves: a fill valve 2202, a flush valve 2203, and a flow valve 2204.

In operation, the piston 2104 within the piston chamber 2103 acts as a piston pump. As the piston 2104 retracts, fluid is drawn into the piston chamber from the fluid supply 3020. No fluid can be drawn from the oxygen chamber passageway because valve assembly is closed and a check valve 2102 is closed in this direction. As the piston 2104 is pushed upward, the fluid within the piston chamber 2103 is pressurized, typically to about 700 psig, and expelled from the piston chamber through the fluid passageway.

The size and shape of the cartridge 2100, the contour of the cartridge housing 2050 (see FIG. 3), and the closing of the housing door 2051 ensure that the cartridge is positioned in a desired manner within the cartridge housing. Correct positioning is important due to the placement of the needle valves and vent valves 2107, shown in FIG. 4 of the cartridge 2100 and the manner in which they are controlled and actuated. The needle valves and vent valves 2107 of the cartridge 2100 are actuated by the cartridge housing. The top of the cartridge 2100 includes two vent valves 2107, and the bottom of the cartridge includes three needle valves. These valves are electromechanically actuated using solenoid-actuated pins.

Oxygen is delivered under pressure to the oxygen chamber 2105 via a passageway. The oxygen supply 1022 (not shown) is coupled to the inlet of the passageway to provide the desired oxygen supply. If all of the needle valves are closed, fluid flows around the closed fill valve 2202 and into a passageway that leads to a nozzle 2108. The nozzle includes a central passageway in which a one-way valve is disposed. The one-way valve is a check valve 2102.

The nozzle 2108 forms fluid droplets into which the oxygen within the oxygen chamber 2105 diffuses as the droplets travel within the oxygen chamber. This oxygen-enriched fluid is referred to as supersaturated oxygen (SSO₂) solution. The droplets infused with the oxygen fall into a pool at the bottom of the oxygen chamber 2105. Since the nozzle 2108 will not atomize properly if the level of the pool rises above the level of the nozzle, the level of the pool is controlled to ensure that the nozzle continues to function properly.

By design, oxygen is dissolved within the atomized fluid to a much greater extent than fluid delivered to the oxygen chamber 2105 in a non-atomized form. Preferably, the oxygen chamber operates at a pressure that is greater than 100 psig. In one embodiment, the oxygen chamber operates at a pressure of about 600 psig. Operating the oxygen chamber at 600 psig, or any pressure above 100 psig promotes finer droplet formation of the physiologic solution from the nozzle 2108 and better saturation efficiency of the gas in the physiologic fluid than operation at a pressure below 100 psig. The oxygen-supersaturated fluid formed within the oxygen chamber 2105 is delivered to the mixing chamber 2106 where it is combined with the blood from the patient. Because it is desirable to control the extent to which the patient's blood is enriched with oxygen, and to operate the system at a constant blood flow rate, it may be desirable to dilute the oxygen-supersaturated fluid within the oxygen chamber 2105 to reduce its oxygen content. When such dilution is desired, the fill valve 2202 is opened to provide a relatively low resistance path for the fluid as compared to the path through the nozzle 2108. Accordingly, instead of passing through the nozzle, the fluid flows through a passageway which extends upwardly into the oxygen chamber via a tube. The tube is advantageously angled somewhat tangentially with respect to the cylindrical wall of the oxygen chamber 2105 so that the fluid readily mixes with the oxygen-supersaturated fluid in the pool at the bottom of the oxygen chamber.

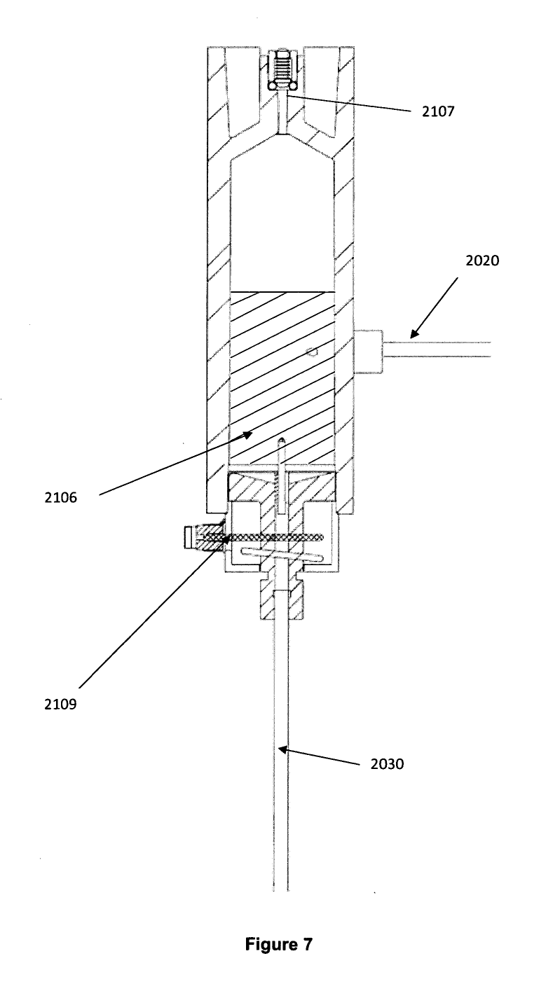

The valve assembly (fluid manifold) essentially performs two additional functions. First, with the fill valve 2202 and the flow valve 2204 closed, the flush valve 2203 may be opened so that fluid flows from the inlet passageway can pass through a series of passageways, the latter of which has a much larger cross-sectional area larger than the cross-sectional area of the flow valve 2204. Thus, the fluid preferentially flows through the larger outlet passageway that is coupled to a capillary tube 2109 seen on FIG. 7. The capillary tube terminates in a tip that extends upwardly into the mixing chamber 2106. Since this fluid has not been gas-enriched, it essentially serves to flush the passageways and the capillary tube 2109. Second, referring back to FIG. 4, with the fill valve 2202 and the flush valve 2203 closed, the flow valve 2204 may be opened when it is desired to deliver the gas-supersaturated fluid from the pool at the bottom of the oxygen chamber 2105 into the mixing chamber 2106.

Referring to FIG. 7, the gas-supersaturated fluid readily flows from the oxygen chamber through the capillary tube 2109 and into the mixing chamber when the flow valve 2204 is open (not shown) due to the fact that pressure within the oxygen chamber 2105 (not shown) is relatively high, e.g., approximately 600 psig, and pressure within the mixing chamber 2106 is relatively low, e.g., about 20 psig. The end of the capillary tip is positioned below a blood inlet of the mixing chamber 2106. This spacial arrangement typically ensures that the blood flowing through the draw tube 2020 and into the blood inlet effectively mixes with the oxygen-supersaturated fluid flowing into the mixing chamber 2106 through the capillary tip. Finally, by the force of the fluid pump the oxygenated blood is pumped out of the mixing chamber through an outlet into the return tube 2030.

Typically, the capillary tube 2109 and the capillary tip are relatively long to ensure that proper resistance is maintained so that the oxygen within the oxygen-supersaturated fluid remains in solution as it travels from the oxygen chamber 2105 into the mixing chamber. The capillary tube and the tip are in the range of 50 microns to 300 microns in length and in the range of 3 inches to 20 inches in internal diameter. To maintain the compact size of the gas enrichment device, therefore, the capillary tube 2109 is coiled around the base of the mixing chamber, as illustrated in the detailed drawing of FIG. 7.

To protect the coiled capillary tube from damage, a protective shield is advantageously formed around the coiled capillary tube to create a compartment.

The orientation and construction of the cartridge 2100 and return tube 2030 shown in FIG. 4 may be of particular importance in light of the fact that the gas-enriched bodily fluid may be gas-saturated or gas-supersaturated. The extracorporeal circuit of the present invention is designed to greatly minimize the potential for larger bubbles (greater than 1000 microns) to proceed through the fluid path to the bubble detector. First, the mixing chamber 2106 is a vertical cylinder with a tangential inlet and an axial outlet. The vortical flow (swirl) induced by the tangential inlet enhances mixing but also enhances de-bubbling of the gas-supersaturated mixture. Second, the mixing chamber exit is at the bottom of the mixing chamber, further inhibiting the flow of bubbles downward into the return tube. Further, the return tube 2030, is designed to reduce or eliminate the creation of cavitation nuclei which may cause a portion of the gas to come out of gas supersaturated solutions. For instance, the diameter of the return tube 2030 is selected to create laminar flow characteristics from the cartridge to the patient, including through the ultrasonic probe. Both the oxygen chamber 2105 and the mixing chamber 2106 include vent valves 2107. As previously mentioned, the cartridge 2100 may optionally include an information recording element for recording data relevant to a procedure, such as flow time, desired concentration, etc. The recording element may be any suitable information recording device such as a bar code label, an RFID chip, a PROM, a flash memory, or any other suitable memory device commonly used in the art. The information recording element may also be used to record relevant patient information such as patient information (e.g., age, sex, weight, height, etc.), procedural data, and specific system setup information tailored to the receiving patient's treatment plan. Inclusion of such information further enhances operator convenience and patient safety.

When information recording elements are included in the cartridge 2100, the system may further include corresponding means to retrieve and utilize the information. For example, if a bar code label is used, the system may further include a bar code reader. The on-board system controller may further include an internal database or be connected to an external information system for retrieving information corresponding to the bar code. When the information includes operating parameters such as treatment duration, temperature, concentration, flow rate, etc., they may be automatically utilized by the system controller.

Alternatively, the information retrieving means may be a separate stand-alone system to be used in conjunction with the gas enrichment system of the present invention. For example, a stand-along bar code reader may be used to read the bar code on the cartridge 2100 by the operator prior to inserting the cartridge into the system.

Priming the Fluid Path

As discussed above, the cartridge 2100 of the present preferred embodiment has a three-chambered body. Referring to FIG. 4, the cartridge 2100, when loaded into the cartridge housing 2050 (shown in FIG. 3), forms a number of fluid pathways, notably the extracorporeal circuit pathway, which conducts bodily fluid from the patient through the draw tube 2020 into the mixing chamber, and then returning the bodily fluid to the patient via the return tube 2030, and the physiologic fluid pathway, which draws the physiologic fluid from the physiologic fluid supply 3020 into the piston chamber 2103 to be pressurized and transmitted to the oxygen chamber 2105 and then directed into the mixing chamber to be mixed with the bodily fluid. Prior to using the system and the cartridge, the various segments of the fluid paths must be primed with an appropriate fluid.

The piston chamber 2103 and the oxygen chamber 2105 must be properly primed with the physiologic fluid before beginning of SSO₂ administration, whereas the draw tube, the mixing chamber, and the return tube must be properly primed with the bodily fluid before SSO₂ administration. It is an advantageous feature of the present invention that these priming steps are automated by the system controller. The general steps of priming the fluid pathways are outlined as follows:

Referring back to FIG. 3, when the cartridge is properly loaded into the cartridge housing 2050, and the housing door 2051 is closed, the system begins the preparatory steps of priming the appropriate cartridge chambers with the physiologic fluid. During this stage, the system displays a progress message on the LCD display to indicate that preparatory procedures are in progress. During the preparatory procedures, the system also performs a series of diagnostic checks to ensure that the cartridge and the system are operating normally. Once the system is finished with the initial preparatory procedures, a message is displayed to indicate to the user that priming of the extracorporeal circuit may be initiated.

The user may then connect the extracorporeal circuit by mounting the draw tube 2020 in position through the fluid pump head. The return tube 2030 is also mounted in position through the ultrasonic probe 2060 and the return clamp 2070 but is not yet connected to the patient via a catheter. At this stage, the return clamp 2070 is closed to effectively seal off any fluid from exiting the return tube 2030. These steps may be performed either while the system is performing the initial preparatory steps, or after the system indicates that initial preparatory steps are completed.

Once the system has completed the initial preparatory steps and the user has connected the extracorporeal circuit, the user may then begin priming the extracorporeal circuit by pressing the priming switch 3040 on display module 3000 (not shown). It is an advantageous feature of the present invention that the extracorporeal path priming is automated with built-in safety checks without requiring user intervention. During the first stage of extracorporeal circuit priming, bodily fluid is drawn into the mixing chamber through the draw tube while the return clamp 2070 and the vent valve 2107 of the mixing chamber (refer to FIG. 4) are held closed. As the bodily fluid fills the chamber, hydrostatic pressure will build up in the pathway to verify that all components are properly connected with no leakage in the pathway. The cartridge pressure sensor 2040 measures the pressure within the extracorporeal circuit. When a proper pressure is reached (approximately 5 psi), the mixing chamber vent valve is opened to allow fluid level to be established. The level sensor in the mixing chamber continues to monitor the level of the bodily fluid in the chamber until the fluid has reached a level appropriate for mixing action to commence. At this point, the return clamp 2070 is released and the fluid is allowed to exit the return tube 2030.

After a small amount of bodily fluid has exited the return tube 2030 to establish a constant flow rate and pressure, the user verifies that no visible bubble is present in the exiting fluid. Then, the user makes wet-to-wet connection between the return tube 2030 and the SSO₂ delivery catheter 4000 shown in FIG. 5 to complete the extracorporeal circuit. In this way, bubble formation is minimized as a result of the fluid pathway design and the priming procedures. No independent or external bubble eliminator is needed in a system of the present invention. Once the system detects that the wet-to-wet connection has been made due to the appropriate increase in return pressure, the bubble detector/flow meter functionality of the ultrasonic probe 2060 is enabled.

Extracorporeal circuit occlusion detection and recovery

It will be noted that one advantageous feature of the present invention is the automated safety responses enabled by the system controller 2080 and its safety interlock 2090 shown in FIG. 1. In conventional extracorporeal systems, occlusion in the fluid conduits such as tubes or catheters can occur. Vigilant monitoring by the human operator is often required to detect such occlusion events and resolve them by manually stopping the system, removing the occlusion, and then restarting the system regardless of the degree of the occlusion. This indiscriminate occlusion resolution procedure can be both time consuming and labor intensive. Because systems of the present invention have a built-in system controller/safety interlock, it is possible for the system to monitor for occlusion events by measuring changes in flow rate, bubble activity, and/or extracorporeal circuit pressure, and respond according to the level of occlusion. For example, if the occlusion event is minor and temporary, the system controller may respond by stopping SSO₂ solution infusion, allowing continued extracorporeal circulation, and notifying the operator to restart SSO₂ solution infusion without requiring a complete shutdown of the system. In the event that the occlusion is a major and continuous, the system controller may respond by stopping SSO₂ solution infusion, stopping extracorporeal circulation, and notifying the operator to resolve the issue before restarting extracorporeal circulation and SSO₂ solution infusion. The system controller also can inform the user of the location of the occlusion (draw tube or return tube) based on pressure and flow information and alert the user accordingly.

Combination Bubble-Detector/Flow Meter

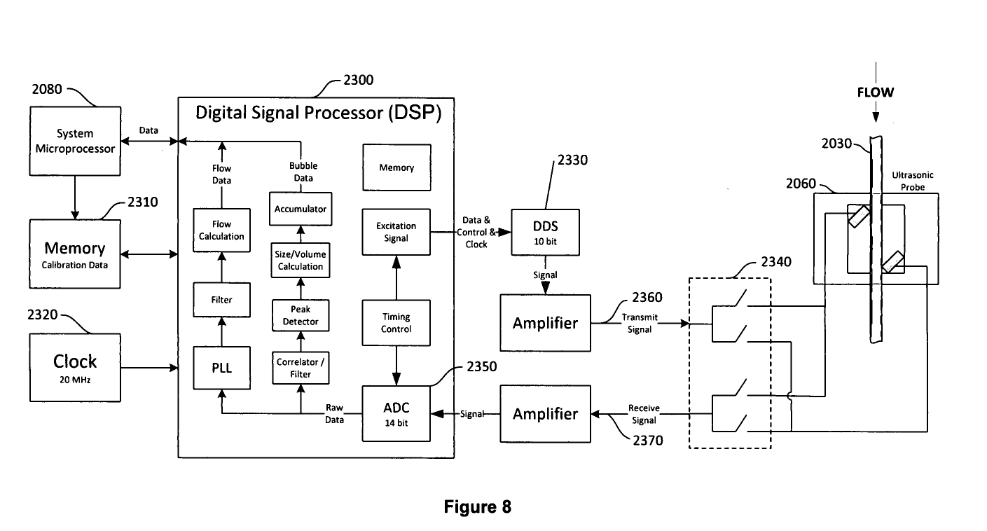

The system of the present invention advantageously includes a bubble detector/flow meter to monitor the oxygen-enriched blood in the return tube 2030 for presence of bubbles and fluid flow rate. The bubble detector/flow meter functionality is provided by an ultrasonic probe 2060 whose signal can simultaneously be monitored for bubble measurement and flow measurement. As shown in FIGS. 1, 2, and 3 of the present preferred embodiment, the ultrasonic probe 2060 is contained within the Cartridge Control subsystem 2001, mounted on the mid-section control module 2000, and coupled to the return tube 2030. The ultrasonic probe 2060 is operated by a digital signal processor (DSP) 2300 on the system controller 2080 to continuously monitor the return blood path for bubbles and flow rate. A block diagram of the bubble detector/flow meter electronic circuit is described in FIG. 8.

In one embodiment, the ultrasonic probe 2060 includes piezoelectric crystals oriented facing each other across the fluid path. For simplicity, the probe will be described with respect to a single crystal pair, although two or more crystal pairs may be used.

Piezoelectric crystals can be designed to resonate in specific frequency ranges by crystal size, shape, and orientation. For monitoring fluids, ultrasonic frequencies in the range of 1 MHz-5 MHz are commonly used. In one embodiment, the piezoelectric crystals may be designed to resonate at approximately 3.6 MHz. The orientation of the crystal pair to the fluid path depends on the application. For bubble detection, the ideal orientation of the crystal pair is perpendicular to the flow path. However, crystals perpendicular to the flow path are not ideal for flow measurement. In order to perform both bubble detection and flow measurement, a 45-degree angle is suitable, although other angles can be used.

The construction of the ultrasonic probe is not particularly limited, but the probe does need to rigidly maintain the position and orientation of the piezoelectric crystals. The ultrasonic probe also needs to provide a receptacle for positioning and holding the return tube in place to perform bubble detection and flow measurement. Advantageously, an epoxy resin can be used to set the position of the crystals in the ultrasonic probe, and a slot for the return tube can be either molded or machined into the epoxy resin to establish the position of the tubing. Various means, such as a hinged door, can be used to load and capture the return tube in place for bubble and flow measurement.

In operation, the ultrasonic probe 2060 uses one crystal as a transmitter and the other as a receiver. For bubble detection, a probe can use one crystal as the transmitter and one as the receiver. For flow measurement, crystals are used alternately as transmitter and receiver, so that the resulting time-of-flight difference between upstream and downstream transit times can be used to measure flow rate (an upstream signal travels against fluid flow, whereas a downstream signal travels in the same relative direction as fluid flow). In order to perform both bubble detection and flow measurement, the crystals can be alternated as transmitters and receivers with no impact on bubble detection.

The DSP 2300 is the processor whose software controls the generation of input signal to the ultrasonic probe 2060 and whose software captures and processes the return signal from the probe. The DSP operation can be initiated (booted) and commanded by the system controller 2080 using an external memory (volatile or non-volatile) such as flash memory 2310. Once initiated, the DSP software can send data to and receive commands from the microprocessor of the system controller 2080.

In order to perform both bubble measurement and flow measurement, precise timing of signal generation and signal capture is essential. Advantageously, the DSP can use a single clock 2320 to control timing of signal generation by the Direct Digital Synthesizer (DDS) 2330, signal path switching by the analog switches 2340, and signal capture by the Analog to Digital Converter (ADC) 2350. Both transmit and receive analog switch timing and signal routing directions are controlled by signals from the DSP. After the receive signal has been converted into the digital domain by the ADC 2350, the DSP software performs bubble detection and flow measurement using the converted signal data.

Transmit Signal Generation

The DSP software controls the timing of the transmit signal generation. The DSP software commands the DDS 2330 to generate the transmit (excitation) signal for the ultrasonic probe 2060. The transmit signal is defined and controlled by the DSP 2300, sending waveform definition, frequency, and timing to the DDS 2330. The DDS 2330 can generate a stepped sinusoidal signal, square wave signal, or other signal forms. The signal may include a single pulse or a series of pulses at various frequencies. In one embodiment of the present invention, the signal generated by the DDS 2230 is a stepped sinusoidal signal and has twenty pulses (cycles) at a 3.6 MHz frequency. The signal has a 10 KHz (100 microsecond) repetition rate that is generated by the DDS 2330 using the data, control, and clock signals from the DSP 2300. The stepped sign wave can be high pass filtered, amplified, and transmitted on signal line 2360. The signal is gated to the transmitter of the crystal pair by the transmit section of the analog switch 2340. The transmitted signal can be either single-ended (referenced to ground) or differential (not referenced to ground), Advantageously, a 10 V differential signal is used (+/−5 Volt peaks), although other voltages could be used. The transmit signal propagates from the transmitting crystal through the tubing containing the flowing solution and generates a receiving signal by exciting the receiving crystal.

Receive Signal Data Capture

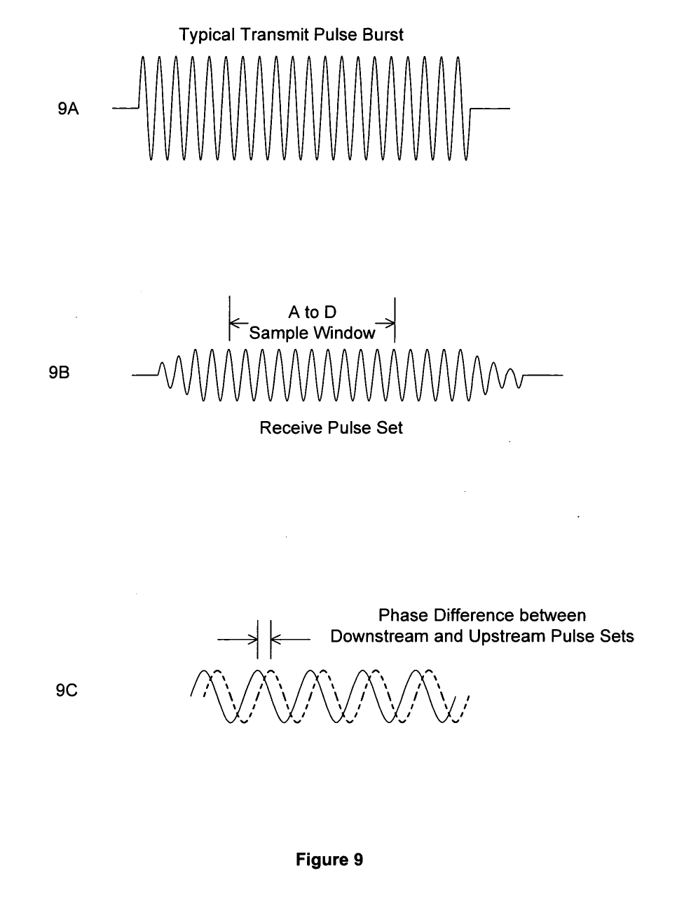

As illustrated in FIG. 9A, the transmitted signal delivered by the ultrasonic probe 2060 typically includes bursts of high frequency pulses. For example, each pulse burst may include 20 pulses at 3.6 MHz, with 100 microseconds between bursts. The return signal from the opposite crystal of the ultrasonic probe 2060 is received on the signal line 2370. The signal received from the ultrasonic probe 2060 on signal line 2370 resembles the transmitted signal, but its phase is shifted and its signal has a smaller amplitude. The receive signal is routed to an amplifier via the receive section of the analog switch (see FIG. 9B). The amplifier high pass filters, amplifies, and shifts the analog signal for input to the ADC 2350. In one embodiment, the signal is amplified and shifted to a 0 to 2 V signal range. Every 100 microseconds, each analog signal pulse is sampled several times by the ADC. In one embodiment, the ADC captures at least 30 samples of each received signal pulse and stores the data in the DSP memory. Since the DSP controls the timing of both transmitted signals and data capture, it identifies captured data as either upstream or downstream data for use by the DSP software.

Bubble Detection and Measurement

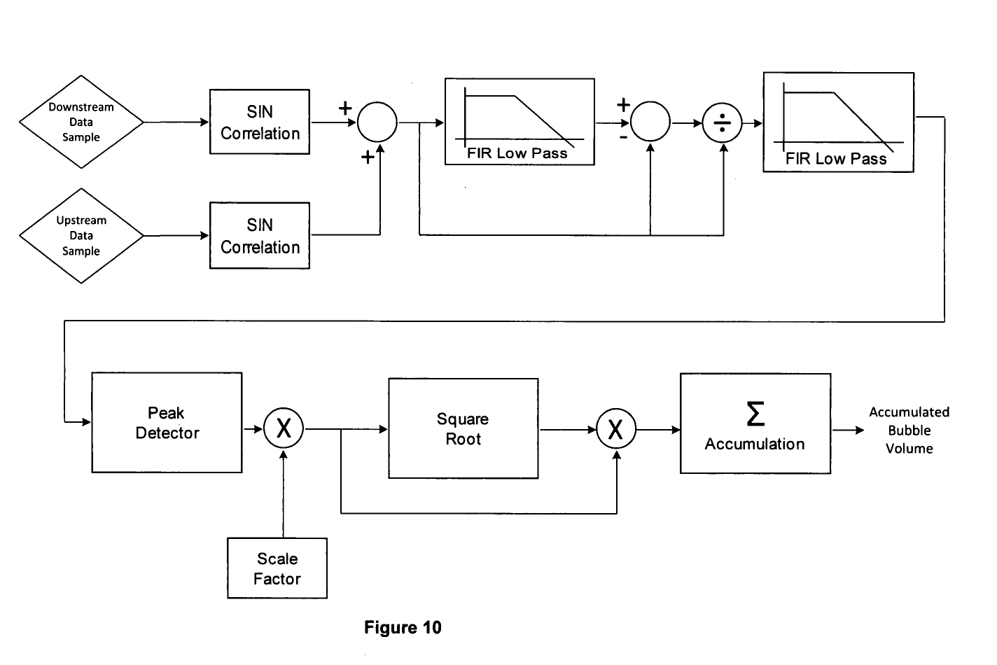

The bubble detection and measurement process involves using captured signal values to calculate the signal amplitudes, average them, check the signal level against a lower limit threshold, peak detect the signal, and determine the end of the bubble state. The bubble diameter is determined using the bubble peak and end of bubble data. The bubble area is then calculated and used to determine the bubble volume. The bubble volume is added to a cumulative bubble volume by the DSP software and both individual volume and accumulated volume are sent to the system controller 2080. This process is represented in FIG. 10.

The strength of a received signal on line 2370 relative to the other received signals on the line 2370 provides information regarding the presence of bubbles within the return tube 2030. The ultrasonic signal from the transmitter crystal is transmitted through the return tube 2030, as well as any fluid within the return tube 2030, to the receiver crystal. If the fluid in the return tube 2030 contains no bubbles, the ultrasonic signal propagates from the transmitter to the receiver in a relatively efficient manner. Thus, the signal strength of the return signal on the line 2370 is relatively strong. However, if the fluid within the return tube 2030 contains bubbles, the ultrasonic signal received will be attenuated. The poorer transmission of the ultrasonic signal across fluid containing bubbles results from the fact that the bubbles tend to scatter the ultrasonic signal. The attenuation in the peak signal is proportional of the projected area of the bubble passing through the probe 2060 at the time the signal was transmitted. Therefore, the amount of the reduction in the signal is proportional to the square of the bubble diameter, so that the square root of the signal is directly proportional to the size of the bubble. This relationship between signal attenuation and bubble size allows for the calibration of the instrument to directly measure the size of entrained bubbles. The ultrasonic probe 2060 used in the present preferred embodiment is capable of measuring and counting individual bubbles smaller than 100-gm in diameter, but other probes could be used. The 100-gm diameter is set as a minimum size for bubble volume measurement and accumulation because bubbles less than this size have negligible volumes less than one nanoliter.

The DSP 2300 software bubble detection process shown in FIG. 10 can use both upstream and downstream signal data to detect bubbles. The data are converted to numbers representing signal amplitude and then signal amplitude is averaged to help filter noise. The signal amplitude average is filtered using a low-pass filter such as a Finite Impulse Response (FIR) filter, and low frequency (DC) signal components are removed using a high-pass filter to ensure that bubbles are discriminated from noise in the signal. The filtered amplitude average is then checked to determine if amplitude variations are greater than a bubble detection noise threshold. If the signal exceeds the bubble detection threshold, indicating a measurable bubble, the sample by sample peak amplitude variation is calculated and each peak is added to a bubble counter as a bubble. The threshold for bubble detection can be calibrated and set by DSP software. A peak is qualified when either the signal drops below the noise threshold, or reaches a relative minimum, and then rises above the relative minimum plus the noise threshold. The peak detection scheme is designed to respond to bubbles that are closely spaced, or even overlapping in the sensor. Overlapping bubbles produce a longer signal drop and have slightly overestimated volumes versus separate bubbles, which produces a conservative cumulative bubble volume. It takes longer than one signal burst period for a bubble to pass through the ultrasonic probe. Each bubble will be sampled by many separate signal pulses as it travels through the ultrasonic probe 2030.

Once a bubble peak has been captured the resulting peak is first scaled to reflect a normalized area, because the measured ultrasonic signal attenuation is proportional to the projected area of the bubble. The relationship between signal attenuation and bubble area can be calibrated and set by DSP software. The area is then divided by pi (π) and the square root is calculated to obtain the bubble radius. The radius is then multiplied by the scaled area and then by 4/3 to generate the bubble volume. However, it should be understood that the volume of the bubble delivered increases within the return tube 2030 as the pressure decreases toward the delivery point, in accordance with the Ideal Gas Law.

Because the pressure of the fluid within the return tube 2030 is typically higher, e.g., approximately two to three atmospheres, as compared to the blood within the patient's vessels, e.g., approximately one atmosphere, a conversion is advantageously performed to determine the volume of the bubble at atmospheric pressure. Since the pressure in the return tube 2030 is monitored by the system via a cartridge pressure sensor cable 2062, and since the pressure of the patient's blood can be assumed to be about atmospheric pressure, the volume of the bubble at atmospheric pressure equals Vₐ=(Pₚ·Vₚ)/Pₐ, where Vₐ is the volume of the bubble at the atmospheric pressure, Pₚ is the pressure at the probe 2030, Vₚ is the volume of the bubble at the probe 2030, and Pₐ is atmospheric pressure.

The DSP software adds the individual bubble volumes to an accumulated bubble volume so that the total amount of infused gas is monitored throughout the procedure. The accumulated bubble volume is transmitted to the system controller 2080. for further processing. If the cumulative bubble volume reaches an established threshold (for example 10 μl) during the treatment or if the probe signal strength is out of range, the system controller 2080 initiates a shutdown for safety reasons.

Flow Measurement

In one embodiment of the present invention, the ultrasonic probe has a pair of crystals oriented across the fluid path at a 45-degree angle. The signal can be sent in either direction, upstream (signal traveling in opposition to fluid flow) or downstream (signal traveling in same direction as fluid flow). After data has been collected by the DSP ADC, the DSP software derives the flow rate by calculating transit time difference between the upstream and downstream signal phases. The relationship between transit time difference and fluid flow rate can be calibrated and set by DSP software.

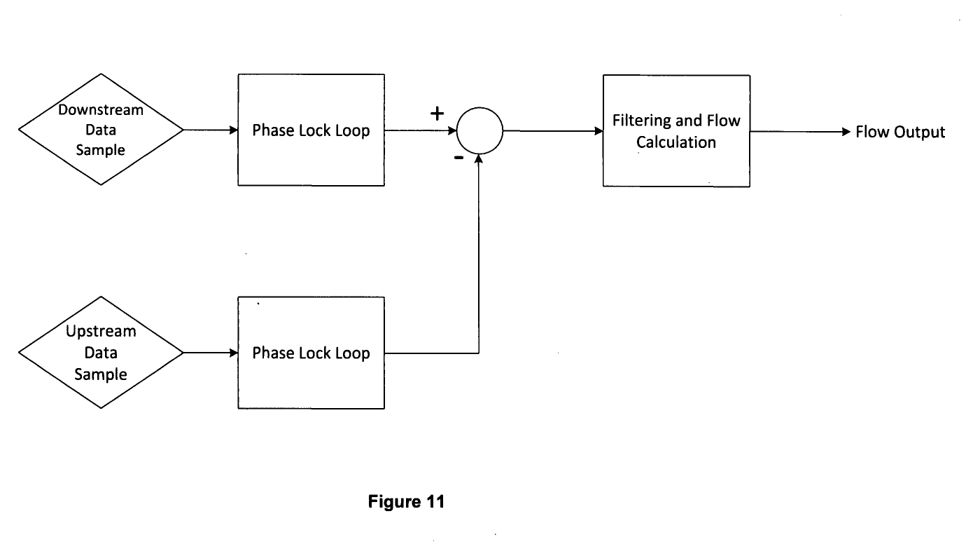

The flow rate is proportional to the differences between the two phase angles as depicted in FIG. 9C, so time-of-flight measurements can be done by calibrating the phase angle differences against a known flow rate and then using the proportionality to determine other flow rates. Detection range can be between 1 to 200 ml/min. FIG. 11 is a block diagram illustrating the algorithm in accordance with the present invention for flow calculation. The procedure to calculate the flow rate first calculates the upstream and downstream phases by running a phase lock loop algorithm against the sampled data. Data for each signal pulse (captured every 100 microseconds) is sampled at least 30 times (at a 10 MHz rate) to reproduce the captured wave form. The phase lock loop builds sine and cosine tables to calculate the received signals in-phase and quadrature phase signal wave forms. Using a Proportional-Integral feedback system, the quadrature phase term is driven to zero. The value required to keep the quadrature phase at zero is a phase angle term used to calculate flow results. The output of the phase lock loop determines the optimal phase angle to the captured wave form. Once the phase angle of each signal pulse has been determined, the difference between upstream and downstream phase angles is calculated and the signal is adjusted by a calibration factor to determine the flow rate.

Subtle variations in the ultrasonic probe crystals and other physical dimensions can influence flow measurement accuracy, but these differences can be minimized by calibration of the probe. Other factors which influence flow measurement accuracy include DSP software algorithms (specifically filtering and averaging) ADC precision, clock frequency (especially sample rate), DSP clock jitter, DDS clock jitter, and ADC clock jitter. Due to the sample rate (10 MHz) and the precision of the ADC (48-bit resolution), these factors have very small influence on the accuracy of flow measurement. Clock jitter, particularly when measuring very small transit time differences (on the order of picoseconds) becomes a larger error component. Advantageously, a common clock reference can be used for the DSP clock, DDS clock and ADC clock, and this clock can be specified to have minimal jitter (less than one picoseconds). The result of this arrangement is that flow can be measured to a precision of +/−1 ml/min in the embodiment described herein.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A gas-enrichment system for enriching a bodily fluid with a gas-enriched fluid in an extracorporeal circuit, comprising:
a combination bubble detector/flow meter comprising an ultrasonic probe that is capable of generating a single ultrasonic signal for measuring both the flow rate and bubbles of the gas-enriched bodily fluid in the system; and
a signal processing unit for processing the single ultrasonic signal generated from the ultrasonic probe to determine the flow rate and measure the bubbles.

2. The system of claim 1, wherein said ultrasonic probe comprises at least one pair of transmitter and receiver, positioned on the opposite side of the fluid path across from each other, for transmitting and receiving ultrasound signals respectively.

3. The system of claim 2, wherein one of the transmitter receiver pair functions as a transmitter for transmitting ultrasound signals in one direction, and as a receiver for receiving ultrasound signals in the opposite direction, while the another of the transmitter receiver pair functions as a receiver for receiving ultrasound signals in one direction, and as a transmitter for transmitting ultrasound signals in the opposite direction.

4. The system of claim 3, wherein said transmitter and receiver pair is positioned such that one is upstream of the other relative to the direction of fluid flow in the fluid path.

5. The system of claim 4, wherein said transmitter and receiver pair is positioned at approximately 45 degrees relative to the direction of fluid flow.

6. The system of claim 4, wherein said flow rate is determined based on the difference between the time-of-flight measured between one of the transmitter receiver pair in the upstream direction versus another of the transmitter receiver pair in the downstream direction.

7. The system of claim 6, wherein the flow rate is from 0-150 ml/min., and the measurement accuracy is within 10 ml/min.

8. The system of claim 1, wherein said bubble is measured based on attenuation of the average amplitude of the signal measured at each receiver, with active corrections for distortion of said signal.

9. The system of claim 8, wherein the bubble measurement is capable of detecting and measuring microbubbles less than 1000 microns in size.

10. The system of claim 8, wherein the bubble measurement is capable of detecting and measuring microbubbles less than 500 microns in size.

11. The system of claim 1 further comprises a system controller that monitors the flow rate, the bubbles, and a circuit pressure for detecting and responding to extracorporeal circuit occlusions.

12. A method for measuring flow rate and bubbles in a fluid along a fluid path utilizing a single ultrasonic signal in an extracorporeal circuit of a gas-enrichment system, the method comprising:
providing an ultrasonic probe for generating a probing ultrasonic signal directed at the fluid;
transmitting the probing signal to the fluid;
receiving a return signal from the fluid;
de-convoluting the return signal into a bubble signal component and a flow rate signal component; and
measuring flow rate and bubbles based on the de-convoluted return signal.

13. The method of claim 12, wherein said ultrasonic probe comprises at least one pair of transmitter and receiver, positioned on the opposite side of the fluid path across from each other, for transmitting and receiving ultrasound signals respectively.

14. The method of claim 13, wherein one of the transmitter receiver pair functions as a transmitter for transmitting ultrasound signals in one direction, and as a receiver for receiving ultrasound signals in the opposite direction, while the another of the transmitter receiver pair functions as a receiver for receiving ultrasound signals in one direction, and as a transmitter for transmitting ultrasound signals in the opposite direction.

15. The method of claim 14, wherein said transmitter and receiver pair is positioned such that one is upstream of the other relative to the direction of fluid flow in the fluid path.

16. The method of claim 15, wherein said transmitter and receiver pair is positioned at approximately 45 degrees relative to the direction of fluid flow.

17. The method of claim 12, wherein said de-convoluting step comprises:
transforming the return signal into a flow rate measurement based on a time-of-flight difference measured between one of the transmitter receiver pair in the upstream direction versus another of the transmitter receiver pair in the downstream direction.
transforming the return signal into a bubble measurement based on signal amplitudes measured at each receiver.

18. The method of claim 17, wherein the flow rate measurement is from 0-150 ml/min with an accuracy of within 10 ml/min.

19. The method of claim 17, wherein the bubble measurement is capable of detecting and measuring microbubbles less than 1000 microns in size.

20. The method of claim 12, further comprising the steps of measuring the circuit pressure of the gas-enrichment system, and monitoring the flow rate, the bubbles, and the circuit pressure for detecting and responding to extracorporeal circuit occlusions.
